(19) United States
(12) Patent Application Publication
(10) Pub. No.: US 2011/0190347 A1
(43) Pub. Date: Aug. 4, 2011

(54) METHODS FOR TREATING NEUROPATHIC PAIN

(75) Inventor: Allyson Gage, West New York, NJ (US)

(73) Assignee: RICHTER GEDEON NYRT., Hungary (unknown)

(21) Appl. No.: 13/059,935

(22) PCT Filed: Aug. 21, 2009
(86) PCT No.: PCT/US09/54563

§ 371 Date: Apr. 25, 2011

Related U.S. Application Data
(60) Provisional application No. 61/090,632, filed on Aug. 21, 2008.

(51) Int. Cl. A61K 31/454; A61P 25/00; A61P 29/00; A61P 19/02; A61P 25/24; A61P 21/02; A61P 25/08; A61P 25/20

(52) U.S. Cl. 514/321

(57) ABSTRACT

The present invention relates to methods of treating diabetic neuropathic pain comprising administering piperidine derivatives, such as 2-[4-(4-fluoro-benzyl)-piperidine-1-yl]-2-oxo-N-(2-oxo-2,3-dihydro-benzoxazol-6-yl)acetamide, and pharmaceutically acceptable salts thereof. Methods of treating post-herpetic neuralgia, chronic lower back pain, osteoarthritis and acute inflammatory pain are described.

### FIELD OF THE INVENTION

The present invention relates to methods for treating neuropathic pain by administering piperidine derivatives, e.g., 2-[4-(4-fluoro-benzyl)-piperidine-1-yl]-2-oxo-N-(2-oxo-2,3-dihydro-benzoxazol-6-yl)acetamide, and pharmaceutically acceptable salts thereof.

### BACKGROUND OF THE INVENTION

Neuropathic pain is a heterogeneous group of neurological conditions that result from damage to the nervous system. Neuropathic pain refers to pain resulting from injury to or dysfunctions of peripheral and/or central sensory pathways, and from dysfunctions of the nervous system, where the pain often occurs or persists without an obvious noxious input. This includes pain related to peripheral neuropathies as well as central neuropathic pain. Central neuropathic pain, involving damage to the brain or spinal cord, can occur following stroke, spinal cord injury, and as a result of multiple sclerosis.

Painful neuropathies are common in patients who have diabetes. It has been estimated that 10-20% of patients with diabetes have chronic neuropathic pain severe enough to require treatment (Boulton, Clin. Diabetes, 23, 9-15, 2005). The pain associated with diabetic neuropathy often leads to comorbidities. Patients who have painful diabetic neuropathies not only experience a diminished quality of life but also incur increased health care costs.

Post-herpetic neuralgia is very common, affecting approximately twenty percent of the entire population of the United States during a lifetime. There may be as many as one million cases in the United States per year and three million cases worldwide in English-speaking, western, and affluent Asiatic countries. Post-herpetic neuralgia is commonly defined as pain that persists or recurs at least one month after occurrence and subsequent healing of herpes zoster in an individual. This pain includes any pain following rash healing to pain persisting for at least three months after rash healing.

Diabetic neuropathic pain and post herpetic neuralgia are distinct disorders that are difficult to treat. For example, the most commonly used pharmacologic agents for diabetic neuropathic pain are anticonvulsants, antidepressants, and opioids, frequently in combination. However, these agents are not effective for all patients, often provide only partial relief of symptoms, and may cause undesirable side effects. Thus, there is an existing and continual need for new pharmaceuticals to treat conditions such as diabetic neuropathic pain and post-herpetic neuralgia, where the drugs are effective for a broader range of patients (particularly for patients resistant to available pharmaceuticals), that are safe and more tolerable, or that complement the efficacy of existing drugs.

### SUMMARY OF THE INVENTION

In one aspect, the present invention relates to methods of treating diabetic neuropathic pain comprising administering piperidine derivatives, such as 2-[4-(4-fluoro-benzyl)-piperidine-1-yl]-2-oxo-N-(2-oxo-2,3-dihydro-benzoxazol-6-yl)acetamide, and pharmaceutically acceptable salts thereof. In other embodiments, methods of treating post-herpetic neuralgia, chronic lower back pain, spinal cord injury, rheumatoid arthritis, osteoarthritis and acute inflammatory pain are described.

### DETAILED DESCRIPTION OF THE INVENTION

In one aspect, the present invention relates to methods of treating diabetic neuropathic pain comprising administering to a patient in need thereof, a therapeutically effective amount of a compound of formula (I):

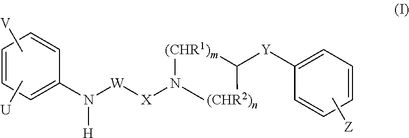

wherein

V and U are each independently

hydrogen, halogen, hydroxyl, cyano, nitro, amino, C₁-C₄ alkylamino optionally substituted by one or more halogen, arylamino optionally substituted by one or more halogen, aralkylamino optionally substituted by one or more halogen, C₁-C₄ alkylsulfonamido optionally substituted by one or more halogen, C₁-C₄ alkanoylamido optionally substituted by one or more halogen, arylsulfonamido, C₁-C₄ alkylsulfonyloxy, carboxyl, trifluoromethyl, trifluoromethoxy, C₁-C₄ alkyl-SO₂—NH—CH₂—, NH₂—(CH₂)₁₋₄—SO₂—NH—, NH₂—(CH₂)₁₋₄—(CO)—NH—, sulfamoyl [NH₂—SO₂—], formyl [—CHO], aminomethyl [—CH₂—NH₂], hydroxymethyl, C₁-C₄ alkyl, C₁-C₄ alkoxymethyl, halogenated methyl, tetrazolyl,

or C₁-C₄ alkoxy, C₁-C₄ alkoxycarbonyl, C₁-C₆ alkanoyloxy, phenyl or C₁-C₄ alkoxy, each of which is optionally substituted by an amino group, or

neighboring V and U groups, together with one or more identical or different additional heteroatoms and/or —CH═ and/or —CH₂— groups optionally form a substituted 4-7 membered homo- or heterocyclic ring (e.g., morpholine, pyrrole, pyrrolidine, oxo-pyrrolidine, thioxo-pyrrolidine, pyrazole, pyrazolidine, imidazole, imidazolidine, oxo-imidazole, thioxo-imidazole, imidazolidine, 1,4-oxazine, oxazole, oxazolidine, oxo-oxazolidine, thioxo-oxazolidine or 3-oxo-1,4-oxazine);

W and X are each independently —CO—, —CH₂— or —CH(C₁-C₄ alkyl)—, with the proviso that W and X can not simultaneously be methylene;

Y is —O—, C₁-C₄ alkylene, C₁-C₄ alkynylene, cycloalkylene, aminocarbonyl, —NH—, —N(C₁-C₄ alkyl)—, —CH₂O—, —CH(OH)— or —OCH₂—;

Z is hydrogen, halogen, nitro, amino, C₁-C₄ alkyl, C₁-C₄ alkoxy, cyano, trifluoromethyl, hydroxyl or carboxy;

R¹ and R² are each independently hydrogen or alkyl, or R¹ and R² together form an optionally substituted C₁-C₃ bridge and

n and m independently are 0-3, with the proviso that n and m can not simultaneously be 0;

and pharmaceutically acceptable salts or solvates (e.g., hydrates) thereof, or solvates of pharmaceutically acceptable salts thereof;

with the further provisos that

when Z is hydrogen, Y is —CH₂—, m and n are 2, R¹ and R² are hydrogen, W is —CO—, X is —CH₂— and V is hydrogen, then U is other than a 4-bromo substituent, and

when Z is hydrogen, Y is —CH₂—, m and n are 2, R¹ and R² are hydrogen, W and X are —CO— and V is hydrogen, then U is other than a 4-carboxyl or 4-ethoxycarbonyl substituent.

In one embodiment, the compound of formula (I) is 2-[4-(4-fluoro-benzyl)-piperidine-1-yl]-2-oxo-N-(2-oxo-2,3-dihydro-benzoxazol-6-yl)acetamide (radiprodil), or a pharmaceutically acceptable salt thereof. The synthesis of radiprodil is described, for example, in U.S. Publication No. 2004/0157886.

In an exemplary embodiment, the present invention relates to a method of treating diabetic neuropathic pain by administering to a patient in need thereof a therapeutically effective amount of 2-[4-(4-fluoro-benzyl)-piperidine-1-yl]-2-oxo-N-(2-oxo-2,3-dihydro-benzoxazol-6-yl)acetamide, or a pharmaceutically acceptable salt thereof.

In one embodiment, the administration of the active ingredient provides therapeutic effects in the treatment of diabetic neuropathic pain (diabetic neuropathy). In one embodiment, the diabetic neuropathic pain is due to diabetes mellitus (e.g., type I or type II diabetes mellitus). In a further embodiment, the administration of the active ingredient provides therapeutic effects in the treatment of diabetic peripheral neuropathic pain (DPNP). In other embodiments, the administration of the active ingredient provides therapeutic effects in the treatment of diabetic autonomic neuropathic pain. In other embodiments, the administration of the active ingredient provides therapeutic effects in the treatment of diabetic proximal neuropathic pain. In other embodiments, the administration of the active ingredient provides therapeutic effects in the treatment of diabetic focal neuropathic pain.

In additional embodiments, the present invention relates to the treatment of neuralgias (e.g., post-herpetic neuralgia) comprising administering a therapeutically effective amount of a compound of formula (I) (e.g., 2-[4-(4-fluoro-benzyl)-piperidine-1-yl]-2-oxo-N-(2-oxo-2,3-dihydro-benzoxazol-6-yl)acetamide) to a patient in need thereof.

In further embodiments, the present invention relates to the treatment of lower back pain (e.g., chronic lower back pain) comprising administering a therapeutically effective amount of a compound of formula (I) (e.g., 2-[4-(4-fluoro-benzyl)-piperidine-1-yl]-2-oxo-N-(2-oxo-2,3-dihydro-benzoxazol-6-yl)acetamide) to a patient in need thereof.

In further embodiments, the present invention relates to the treatment of spinal cord injury comprising administering a therapeutically effective amount of a compound of formula (I) (e.g., 2-[4-(4-fluoro-benzyl)-piperidine-1-yl]-2-oxo-N-(2-oxo-2,3-dihydro-benzoxazol-6-yl)acetamide) to a patient in need thereof.

In other embodiments, the present invention relates to the treatment of rheumatoid arthritis, osteoarthritis or acute inflammatory pain comprising administering a therapeutically effective amount of a compound of formula (I) (e.g., 2-[4-(4-fluoro-benzyl)-piperidine-1-yl]-2-oxo-N-(2-oxo-2,3-dihydro-benzoxazol-6-yl)acetamide) to a patient in need thereof.

In certain embodiments, the compound of formula (I) (e.g., 2-[4-(4-fluoro-benzyl)-piperidine-1-yl]-2-oxo-N-(2-oxo-2,3-dihydro-benzoxazol-6-yl)acetamide) is administered in an amount of between about 0.01 mg and about 150 mg, for example between about 5 mg and about 150 mg, such as between about 10 mg and about 150 mg.

In additional embodiments, the compound of formula (I) (e.g., 2-[4-(4-fluoro-benzyl)-piperidine-1-yl]-2-oxo-N-(2-oxo-2,3-dihydro-benzoxazol-6-yl)acetamide) is administered in an amount of about 1 mg, about 2 mg, about 3 mg, about 4 mg, about 5 mg, about 6 mg, about 7 mg, about 8 mg, about 9 mg, about 10 mg, about 12 mg, about 15 mg, about 18 mg, about 20 mg, about 21 mg, about 24 mg, about 25 mg, about 27 mg, about 30 mg, about 35 mg, about 36 mg, about 40 mg, about 45 mg, about 50 mg, about 54 mg, about 63 mg, about 72 mg, about 75 mg, about 90 mg, about 100 mg, about 108 mg, about 125 mg, about 135 mg or about 150 mg.

For example, the compound of formula (I) (e.g., 2-[4-(4-fluoro-benzyl)-piperidine-1-yl]-2-oxo-N-(2-oxo-2,3-dihydro-benzoxazol-6-yl)acetamide) may be administered in an amount of about 18 mg, about 27 mg, about 36 mg, about 45 mg, about 54 mg, about 63 mg, about 72 mg, about 90 mg, about 108 mg or about 135 mg.

In another embodiment, the present invention relates to a method of treating diabetic neuropathic pain comprising administering to a patient in need thereof a therapeutically effective amount of radiprodil or a pharmaceutically acceptable salt thereof in the dosage amount from about 10 mg to about 150 mg to a patient in need thereof.

In yet another embodiment, the present invention relates to a method of treating diabetic neuropathic pain comprising administering to a patient in need thereof a therapeutically effective amount of radiprodil or a pharmaceutically acceptable salt thereof in the dosage amount of about 18 mg, about 27 mg, about 30 mg, about 36 mg, about 40 mg, about 45 mg, about 50 mg, about 54 mg, about 60 mg, about 63 mg, about 72 mg, about 75 mg, about 80 mg, about 85 mg, about 90 mg, about 95 mg, about 100 mg, about 105 mg, about 108 mg, about 115 mg, about 120 mg, about 125 mg, about 130 mg, about 135 mg, about 140 mg, about 145 mg, or about 150 mg.

In another embodiment, the present invention relates to a method of treating neuralgia's comprising administering to a patient in need thereof a therapeutically effective amount of radiprodil or a pharmaceutically acceptable salt thereof in the dosage amount from about 10 mg to about 150 mg to a patient in need thereof.

In yet another embodiment, the present invention relates to a method of treating neuralgia's comprising administering to a patient in need thereof a therapeutically effective amount of radiprodil or a pharmaceutically acceptable salt thereof in the dosage amount of about 18 mg, about 27 mg, about 30 mg, about 36 mg, about 40 mg, about 45 mg, about 50 mg, about 54 mg, about 60 mg, about 63 mg, about 72 mg, about 75 mg, about 80 mg, about 85 mg, about 90 mg, about 95 mg, about 100 mg, about 105 mg, about 108 mg, about 115 mg, about 120 mg, about 125 mg, about 130 mg, about 135 mg, about 140 mg, about 145 mg, or about 150 mg.

In another embodiment, the present invention relates to a method of treating lower back pain (e.g., chronic lower back pain) comprising administering to a patient in need thereof a therapeutically effective amount of radiprodil or a pharmaceutically acceptable salt thereof in the dosage amount from about 10 mg to about 150 mg to a patient in need thereof.

In yet another embodiment, the present invention relates to a method of treating lower back pain (e.g., chronic lower back pain) comprising administering to a patient in need thereof a therapeutically effective amount of radiprodil or a pharmaceutically acceptable salt thereof in the dosage amount of about 18 mg, about 27 mg, about 30 mg, about 36 mg, about 40 mg, about 45 mg, about 50 mg, about 54 mg, about 60 mg, about 63 mg, about 72 mg, about 75 mg, about 80 mg, about 85 mg, about 90 mg, about 95 mg, about 100 mg, about 105 mg, about 108 mg, about 115 mg, about 120 mg, about 125 mg, about 130 mg, about 135 mg, about 140 mg, about 145 mg, or about 150 mg.

In another embodiment, the present invention relates to a method of treating spinal cord injury comprising administering to a patient in need thereof a therapeutically effective amount of radiprodil or a pharmaceutically acceptable salt thereof in the dosage amount from about 10 mg to about 150 mg to a patient in need thereof.

In yet another embodiment, the present invention relates to a method of treating spinal cord injury comprising administering to a patient in need thereof a therapeutically effective amount of radiprodil or a pharmaceutically acceptable salt thereof in the dosage amount of about 18 mg, about 27 mg, about 30 mg, about 36 mg, about 40 mg, about 45 mg, about 50 mg, about 54 mg, about 60 mg, about 63 mg, about 72 mg, about 75 mg, about 80 mg, about 85 mg, about 90 mg, about 95 mg, about 100 mg, about 105 mg, about 108 mg, about 115 mg, about 120 mg, about 125 mg, about 130 mg, about 135 mg, about 140 mg, about 145 mg, or about 150 mg.

In another embodiment, the present invention relates to a method of treatment of rheumatoid arthritis, osteoarthritis or acute inflammatory pain comprising administering to a patient in need thereof a therapeutically effective amount of radiprodil or a pharmaceutically acceptable salt thereof in the dosage amount from about 10 mg to about 150 mg to a patient in need thereof.

In yet another embodiment, the present invention relates to a method of treatment of rheumatoid arthritis, osteoarthritis or acute inflammatory pain comprising administering to a patient in need thereof a therapeutically effective amount of radiprodil or a pharmaceutically acceptable salt thereof in the dosage amount of about 18 mg, about 27 mg, about 30 mg, about 36 mg, about 40 mg, about 45 mg, about 50 mg, about 54 mg, about 60 mg, about 63 mg, about 72 mg, about 75 mg, about 80 mg, about 85 mg, about 90 mg, about 95 mg, about 100 mg, about 105 mg, about 108 mg, about 115 mg, about 120 mg, about 125 mg, about 130 mg, about 135 mg, about 140 mg, about 145 mg, or about 150 mg.

The desired dose may be administered as one or more daily sub dose(s) administered at appropriate time intervals throughout the day, or alternatively, in a single dose, for example, for morning or evening administration. For example, the daily dosage may be divided into one, into two, into three, or into four divided daily doses. In certain embodiments, the active ingredient is administered in one, two or three (e.g., three) divided daily doses.

In exemplary embodiments, the compound of formula (I) (e.g., 2-[4-(4-fluoro-benzyl)-piperidine-1-yl]-2-oxo-N-(2-oxo-2,3-dihydro-benzoxazol-6-yl)acetamide) may be administered in an amount of about 6 mg TID (about 18 mg/day), about 9 mg TID (about 27 mg/day), about 12 mg TID (about 36 mg/day), about 15 mg TID (about 45 mg/day), about 18 mg TID (about 54 mg/day), about 21 mg TID (about 63 mg/day), about 24 mg TID (about 72 mg/day), about 30 mg TID (about 90 mg/day), about 36 mg TID (about 108 mg/day) or about 45 mg TID (about 135 mg/day).

The duration of the treatment may be decades, years, months, weeks, or days, as long as the benefits persist.

Pharmaceutically acceptable salts include those obtained by reacting the main compound, functioning as a base with an inorganic or organic acid to form a salt, for example, salts of hydrochloric acid, sulfuric acid, phosphoric acid, methane sulfonic acid, camphor sulfonic acid, oxalic acid, maleic acid, succinic acid, citric acid, formic acid, hydrobromic acid, benzoic acid, tartaric acid, fumaric acid, salicylic acid, mandelic acid, and carbonic acid. Pharmaceutically acceptable salts also include those in which the main compound functions as an acid and is reacted with an appropriate base to form, e.g., sodium, potassium, calcium, magnesium, ammonium, and choline salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts can be prepared by reacting the compounds of the invention with the appropriate base via a variety of known methods.

The following are further examples of acid salts that can be obtained by reaction with inorganic or organic acids: acetates, adipates, alginates, citrates, aspartates, benzoates, benzenesulfonates, bisulfates, butyrates, camphorates, digluconates, cyclopentanepropionates, dodecylsulfates, ethanesulfonates, glucoheptanoates, glycerophosphates, hemisulfates, heptanoates, hexanoates, fumarates, hydrobromides, hydroiodides, 2-hydroxy-ethanesulfonates, lactates, maleates, methanesulfonates, nicotinates, 2-naphthalenesulfonates, oxalates, palmoates, pectinates, persulfates, 3-phenylpropionates, picrates, pivalates, propionates, succinates, tartrates, thiocyanates, tosylates, mesylates and undecanoates.

In one embodiment, the pharmaceutically acceptable salt is a hydrochloride salt.

Some of the compounds useful in the present invention can exist in different polymorphic forms. As known in the art, polymorphism is an ability of a compound to crystallize as more than one distinct crystalline or “polymorphic” species. A polymorph is a solid crystalline phase of a compound with at least two different arrangements or polymorphic forms of that compound molecule in the solid state. Polymorphic forms of any given compound are defined by the same chemical formula or composition and are as distinct in chemical structure as crystalline structures of two different chemical compounds. The use of such polymorphs is within the scope of the present invention.

Some of the compounds useful in the present invention can exist in different solvate forms. Solvates of the compounds of the invention may also form when solvent molecules are incorporated into the crystalline lattice structure of the compound molecule during the crystallization process. For example, suitable solvates include hydrates, e.g., monohydrates, dihydrates, sesquihydrates, and hemihydrates. The use of such solvates is within the scope of the present invention.

The compounds of formula (I) can be administered alone as an active ingredient or as an additional ingredient of pharmaceutically acceptable composition.

Numerous standard references are available that describe procedures for preparing various formulations suitable for administering the compounds according to the invention. Examples of potential formulations and preparations are contained, for example, in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Arthur Osol, editor), 1553-1593 (current edition).

The mode of administration and dosage forms is closely related to the therapeutic amounts of the compounds or compositions which are desirable and efficacious for the given treatment application.

Suitable dosage forms include but are not limited to oral, rectal, sub-lingual, mucosal, nasal, ophthalmic, subcutaneous, intramuscular, intravenous, transdermal, spinal, intrathecal, intra-articular, intra-arterial, sub-arachinoid, bronchial, lymphatic, and intra-uterile administration, and other dosage forms for systemic delivery of active ingredients. Formulations suitable for oral administration are preferred.

Various solid oral dosage forms can be used for administering active ingredient including such solid forms as tablets, gelcaps, capsules, caplets, granules, lozenges and bulk powders. In such solid dosage forms the active ingredient is mixed with at least one inert, pharmaceutically acceptable carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quarternary ammonium salts, g) wetting agents such as, for example cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Compositions suitable for buccal or sublingual administration include tablets, lozenges and pastilles, wherein the active ingredient is formulated with a carrier such as sugar and acacia, tragacanth, or gelatin and glycerin.

The solid dosage forms of tablets, capsules, pills and granules can be prepared with coatings and shells such as enteric coatings and other coatings well known in the pharmaceutical formulating art. They may optionally contain opacifying agents and can also be of a composition that they release the crystalline compound of the present invention. In another embodiment of the present invention, radiprodil can be formulated in a time release capsules, tablets and gels which is also advantageous in the targeted release of the crystalline compound of the present invention.

Various liquid oral dosage forms can also be used for administering active ingredient, including aqueous and non-aqueous solutions, emulsions, suspensions, syrups, and elixirs. In addition to the active ingredient, the liquid dosage forms may contain inert diluents commonly used in the art such as, for example, water or other solvents, solubilizing agents and emulsifiers, for example ethyl alcohol, ethyl carbonate, ethyl acetate, propylene glycol, oils, fatty acid esters and mixtures thereof. Besides inert diluents, the oral compositions can also include adjuvants such as wetting agents, emulsifying and suspending agents, sweetening, flavoring and perfuming agents. Aerosol formulations typically comprise typically comprise a solution or fine suspension of the crystalline compound of the present invention in physiologically acceptable aqueous or non-aqueous solvent and are usually presented in single or multidose quantitites in sterile form in a sealed container.

Injectable preparations of the present invention, for example, sterile injectable aqueous or oleaginous suspensions may be formulated according to the known art using suitable dispersing or wetting agents and suspending agents.

Suppositories for rectal administration of the active ingredient can be prepared by mixing the compound with a suitable excipient such as cocoa butter, salicylates and polyethylene glycols. Formulations for vaginal administration can be in the form of a pessary, tampon, cream, gel, past foam, or spray formula containing, in addition to the active ingredient, such suitable carriers as are known in the art.

For topical administration, the pharmaceutical composition can be in the form of creams, ointments, liniments, lotions, emulsions, suspensions, gels, solutions, pastes, powders, sprays, and drops suitable for administration to the skin, eye, ear or nose. Topical administration may also involve transdermal administration via means such as transdermal patches.

Aerosol formulations suitable for administering via inhalation also can be made. For example, for treatment of disorders of the respiratory tract, the active ingredient can be administered by inhalation in the form of a powder (e.g., micronized) or in the form of atomized solutions or suspensions. The aerosol formulation can be placed into a pressurized acceptable propellant.

The invention also provides the use of compounds of formula (I) in the manufacture of a medicament for the treatment of conditions such as diabetic neuropathic pain, post-herpetic neuralgia, chronic lower back pain, osteoarthritis and acute inflammatory pain.

In one embodiment, the compositions of the present invention contain radiprodil between about 0.01% by weight and about 25%, between about 0.05% and about 25%, between about 0.1% and about 25%, between about 0.25% and about 25%, between about 0.5% and about 25%, between about 1% and about 25%, between about 2% and about 20%, between about 4% and about 18%, between about 6% and about 16%, between about 8% and about 14%, between about 10% and about 12% by weight of the pharmaceutically acceptable composition.

To prepare such pharmaceutical dosage forms, the active ingredient is typically mixed with a pharmaceutical carrier according to conventional pharmaceutical compounding techniques. The carrier may take a wide variety of forms depending on the form of preparation desired for administration.

In preparing the compositions in oral dosage form, any of the usual pharmaceutical media may be employed. Thus, for liquid oral preparations, such as, for example, suspensions, elixirs and solutions, suitable carriers and additives include water, glycols, oils, alcohols, flavoring agents, preservatives, coloring agents and the like. For solid oral preparations such as, for example, powders, capsules and tablets, suitable carriers and additives include starches, sugars, diluents, granulating agents, lubricants, binders, disintegrating agents and the like. Due to their ease in administration, tablets and capsules represent the most advantageous oral dosage unit form. If desired, tablets may be sugar coated or enteric coated by standard techniques.

For parenteral formulations, the carrier will usually comprise sterile water, though other ingredients, for example, ingredients that aid solubility or for preservation, may be included. Injectable solutions may also be prepared in which case appropriate stabilizing agents may be employed.

In some applications, it may be advantageous to utilize the active agent in a “vectorized” form, such as by encapsulation of the active agent in a liposome or other encapsulant medium, or by fixation of the active agent, e.g., by covalent bonding, chelation, or associative coordination, on a suitable biomolecule, such as those selected from proteins, lipoproteins, glycoproteins, and polysaccharides.

Treatment methods of the present invention using formulations suitable for oral administration may be presented as discrete units such as capsules, cachets, tablets, or lozenges, each containing a predetermined amount of the active ingredient as, for example, a powder or granules. Optionally, a suspension in an aqueous liquor or a non-aqueous liquid may be employed, such as a syrup, an elixir, an emulsion, or a draught.

A tablet may be made by compression or molding, or wet granulation, optionally with one or more accessory ingredients. Compressed tablets may be prepared by compressing in a suitable machine, with the active compound being in a free-flowing form such as a powder or granules which optionally is mixed with, for example, a binder, disintegrant, lubricant, inert diluent, surface active agent, or discharging agent. Molded tablets comprised of a mixture of the powdered active compound with a suitable carrier may be made by molding in a suitable machine.

A syrup may be made by adding the active compound to a concentrated aqueous solution of a sugar, for example sucrose, to which may also be added any accessory ingredient(s). Such accessory ingredient(s) may include flavorings, suitable preservative, agents to retard crystallization of the sugar, and agents to increase the solubility of any other ingredient, such as a polyhydroxy alcohol, for example glycerol or sorbitol.

Formulations suitable for parenteral administration usually comprise a sterile aqueous preparation of the active compound, which preferably is isotonic with the blood of the recipient (e.g., physiological saline solution). Such formulations may include suspending agents and thickening agents and liposomes or other microparticulate systems which are designed to target the compound to blood components or one or more organs. The formulations may be presented in unit-dose or multi-dose form.

Parenteral administration may comprise any suitable form of systemic delivery. Administration may for example be intravenous, intra-arterial, intrathecal, intramuscular, subcutaneous, intramuscular, intra-abdominal (e.g., intraperitoneal), etc., and may be effected by infusion pumps (external or implantable) or any other suitable means appropriate to the desired administration modality.

Nasal and other mucosal spray formulations (e.g. inhalable forms) can comprise purified aqueous solutions of the active compounds with preservative agents and isotonic agents. Such formulations are preferably adjusted to a pH and isotonic state compatible with the nasal or other mucous membranes. Alternatively, they can be in the form of finely divided solid powders suspended in a gas carrier. Such formulations may be delivered by any suitable means or method, e.g., by nebulizer, atomizer, metered dose inhaler, or the like.

Formulations for rectal administration may be presented as a suppository with a suitable carrier such as cocoa butter, hydrogenated fats, or hydrogenated fatty carboxylic acids.

Transdermal formulations may be prepared by incorporating the active agent in a thixotropic or gelatinous carrier such as a cellulosic medium, e.g., methyl cellulose or hydroxyethyl cellulose, with the resulting formulation then being packed in a transdermal device adapted to be secured in dermal contact with the skin of a wearer.

In addition to the aforementioned ingredients, formulations of this invention may further include one or more accessory ingredient(s) selected from diluents, buffers, flavoring agents, binders, disintegrants, surface active agents, thickeners, lubricants, preservatives (including antioxidants), and the like.

The formulations of the present invention can have immediate release, sustained release, delayed-onset release or any other release profile known to one skilled in the art.

The compound of formula (I) may be adjunctively administered in combination with additional active agents useful in the treatment of pain (e.g., neuropathic pain such as diabetic neuropathic pain, post herpetic neuralgia). For example, the compound of formula (I) may be administered in combination with one or more antidepressants, analgesics, muscle relaxants, anorectics, stimulants, antiepileptic drugs, sedative/hypnotics, and combinations thereof. Specific examples of compounds that can be administered with the compound of formula (I) include, but are not limited to, milnacipran, gabapentin, pregabalin, pramipexole, 1-DOPA, amphetamine, tizanidine, clonidine, tramadol, morphine, tricyclic antidepressants, codeine, carbamazepine, sibutramine, amphetamine, valium, trazodone and combinations thereof (including salts and/or solvates thereof).

By adjunctive administration is meant simultaneous administration of the compounds in the same-dosage form, simultaneous administration in separate dosage forms, and separate administration of the compounds.

In an exemplary embodiment, the compound of formula (I) (e.g., 2-[4-(4-fluoro-benzyl)-piperidine-1-yl]-2-oxo-N-(2-oxo-2,3-dihydro-benzoxazol-6-yl)acetamide), or a pharmaceutically acceptable salt thereof, is administered in combination with milnacipran, or a pharmaceutically acceptable salt thereof (e.g., milnacipran hydrochloride).

### Definitions

The term “pharmaceutically acceptable” means biologically or pharmacologically compatible for in vivo use in animals or humans, and preferably means approved by a regulatory agency of the Federal or a state government or listed in the U.S. Pharmacopeia or other generally recognized pharmacopeia for use in animals, and more particularly in humans.

The terms “treat,” “treatment,” and “treating” refer to one or more of the following:
(a) relieving or alleviating at least one symptom of a disorder in a subject, including for example, diabetic neuropathic pan, post-herpetic neuralgia;
(b) relieving or alleviating the intensity and/or duration of a manifestation of a disorder experienced by a subject including, but not limited to, those that are in response to a given stimulus (e.g., pressure, tissue injury, cold temperature, etc.);
(c) arresting, delaying the onset (i.e., the period prior to clinical manifestation of a disorder) and/or reducing the risk of developing or worsening a disorder.

An “effective amount” means the amount of an active ingredient that, when administered to a patient (e.g., a mammal) for treating a disease, is sufficient to effect such treatment for the disease, or an amount that is sufficient for modulating an NMDA receptor (e.g., NR2B receptor) to achieve the objectives of the invention. The “effective amount” will vary depending on the compound, the disease and its severity and the age, weight, responsiveness, etc., of the patient to be treated.

A subject or patient in whom administration of the therapeutic compound is an effective therapeutic regimen for a disease or disorder is preferably a human, but can be any animal, including a laboratory animal in the context of a trial or screening or activity experiment. Thus, as can be readily appreciated by one of ordinary skill in the art, the methods, compounds and compositions of the present invention are particularly suited to administration to any animal, particularly a mammal, and including, but by no means limited to, humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per practice in the art. Alternatively, “about” with respect to the compositions can mean plus or minus a range of up to 20%, preferably up to 10%, more preferably up to 5%.

### EXAMPLES

The following examples are merely illustrative of the present invention and should not be construed as limiting the scope of the invention in any way as many variations and equivalents that are encompassed by the present invention will become apparent to those skilled in the art upon reading the present disclosure.

### Example 1

This clinical study will be conducted as an in-patient, randomized, double-blind, placebo-controlled study. A total of up to 72 patients will be studied, selected using criteria that include patients clinically diagnosed with painful neuropathy (≧3 months before screening) due to Type I or Type II diabetes mellitus, with a history of diabetes mellitus greater than 1 year.

Up to six cohorts, each with 12 patients randomly assigned to 2-[4-(4-fluoro-benzyl)-piperidine-1-yl]-2-oxo-N-(2-oxo-2,3-dihydro-benzoxazol-6-yl)acetamide (radiprodil) or placebo in a 3:1 ratio will run sequentially. Patients who meet all entry criteria at Screening will be admitted to the investigative site 1 day before the First Dose Day to confirm continued eligibility. Acetaminophen or nonsteroidal anti-inflammatory drugs may be used throughout the study as rescue analgesic medication. Patients who meet all entry requirements at Screening and Baseline (Day -1) will be randomized to double blind escalating doses of radiprodil or placebo (both administered three times a day (TID)). The duration of the double-blind Treatment Phase will vary based on the dosing regimen for each cohort, but it will last no longer than 38 days and will be followed by a 2-day non-treatment PK Sampling Phase.

### Cohorts 1 and 2

The objective for Cohort 1 is to identify the maximum tolerated dose (MTD), and the objective for Cohort 2 is to confirm or extend the MTD. The starting dosage in Cohort 1 will be 6 mg TID (18 mg/day) escalated at 3-day intervals to 45 mg TID (135 mg/day) or MTD according to the fixed dosing regimen given below. Patients randomized to Cohort 1 will receive the following dosages:

Days 1-3 6 mg radiprodil TID (18 mg/day) or placebo
Days 4-6 9 mg radiprodil TID (27 mg/day) or placebo
Days 7-9 12 mg radiprodil TID (36 mg/day) or placebo
Days 10-12 15 mg radiprodil TID (45 mg/day) or placebo
Days 13-15 18 mg radiprodil TID (54 mg/day) or placebo
Days 16-18 21 mg radiprodil TID (63 mg/day) or placebo
Days 19-21 24 mg radiprodil TID (72 mg/day) or placebo
Days 22-24 30 mg radiprodil TID (90 mg/day) or placebo
Days 25-27 36 mg radiprodil TID (108 mg/day) or placebo
Days 28-30 45 mg radiprodil TID (135 mg/day) or placebo
Day 31 45 mg radiprodil in the morning only (45 mg/day) or placebo

The dosing regimen of the next Cohort will be determined after the safety and tolerability of the dosages use in Cohort 1 have been reviewed. Cohort 2, if conducted, will repeat the titration regimen of Cohort 1. The time spent at any dosage may be increased to enhance tolerability. If no MTD is determined in Cohort 1, then Cohort 3 will begin dosing.

### Cohorts 3-5

Based on the safety and tolerability results seen in Cohorts 1 and 2 (if applicable), subsequent Cohorts 3-5 will be used to explore whether tolerability can be enhanced through the use of other titration regimens up to the MTD of 45 mg TID. The dose escalations will range from 3 mg to 15 mg TID (no more frequently than every 2 days) and will not exceed MTD or 45 mg TID. The double-blind Treatment Phase will last no longer than 38 days and will be followed by a 2 day non treatment PK Sampling Phase.

### Cohort 6

The final cohort will repeat the dosing regimen that reaches MTD or 45 mg TID in the shortest period maintenance dose at the MTD or 45 mg TID. The dose escalations will range from 3 mg to 15 mg TID (no more frequently than every 2 days) and will not exceed MTD or 45 mg TID. The double-blind Treatment Phase will last for no longer than 38 days (including the maximum 14-day maintenance at the MTD or 45 mg TID) and will be followed by a 2-day non treatment PK Sampling Phase.

The results from the above treatment regimes may surprisingly show that radiprodil can be used to safely and effectively treat neuropathic pain associated with diabetes mellitus.

### Example 2

A clinical study will be conducted as a multicenter, randomized, double-blind, placebo-controlled study of patients with painful diabetic neuropathy (i.e., patients with a Michigan Neuropathic Screening Instrument score of at least 3). The study will consist of a maximum 5-week screening/washout period (including 1-week of single-blind lead-in treatment) followed by a 17-week double-blind treatment phase (consisting of a 5-week titration period and 12 weeks of stable dosing). This will be followed by a 4-week withdrawal phase.

Patients will be randomized (1:1:1:1:1) to one of 5-double blind treatment groups (low dose radiprodil, medium-dose radiprodil, high-dose radiprodil, comparison drug, placebo).

The results from the above treatment regimes may surprisingly show that radiprodil can be used to safely and effectively treat painful diabetic neuropathy.

### Example 3

A patient with post-herpetic neuralgia presents to a physician's office or clinic. To improve the patient's symptoms, the patient is administered between about 1 and about 150 mg radiprodil per day. The patient's vital signs and an ECG are recorded. Adverse events are also recorded. Physical examinations are conducted and blood and urine samples are collected. At the discretion of the physician, the dosage of radiprodil can be reduced or increased as required. The results from the above treatment regimen may surprisingly show that radiprodil can be used to safely and effectively treat post-herpetic neuralgia.

### Example 4

A patient with chronic lower back pain presents to a physician's office or clinic. To improve the patient's symptoms, the patient is administered between about 1 and about 150 mg radiprodil per day. The patient's vital signs and an ECG are recorded. Adverse events are also recorded. Physical examinations are conducted and blood and urine samples are collected. At the discretion of the physician, the dosage of radiprodil can be reduced or increased as required. The results from the above treatment regimen may surprisingly show that radiprodil can be used to safely and effectively treat chronic lower back pain.

### Example 5

This clinical study will be conducted as a randomized, double-blind, placebo-controlled study in patients who had neuropathic pain due to spinal cord injury.

After a 1-week non-treatment run-in period, patients will be randomized to a treatment sequence of 4 weeks of radiprodil or placebo administered orally, followed by a 1-week wash-out period before the second 4-week treatment with radiprodil or placebo. Patients in the radiprodil treatment period will be titrated over 17 days from a starting dosage of 6 mg TID to a dosage of 15 mg TID.

The results from the above treatment regime may surprisingly show that radiprodil can be used safely and effectively to treat neuropathic pain due to spinal cord injury

### Example 6

A patient with osteoarthritis presents to a physician's office or clinic. To improve the patient's symptoms, the patient is administered between about 1 and about 150 mg radiprodil per day. The patient's vital signs and an ECG are recorded. Adverse events are also recorded. Physical examinations are conducted and blood and urine samples are collected. At the discretion of the physician, the dosage of radiprodil can be reduced or increased as required. The results from the above treatment regimen may surprisingly show that radiprodil can be used to safely and effectively treat osteoarthritis.

### Example 7

A patient with acute inflammatory pain presents to a physician's office or clinic. To improve the patient's symptoms, the patient is administered between about 1 and about 150 mg radiprodil per day. The patient's vital signs and an ECG are recorded. Adverse events are also recorded. Physical examinations are conducted and blood and urine samples are collected. At the discretion of the physician, the dosage of radiprodil can be reduced or increased as required. The results from the above treatment regimen may surprisingly show that radiprodil can be used to safely and effectively treat acute inflammatory pain.

### Example 8

A clinical study will be conducted as a multicenter, randomized, double-blind, placebo-controlled study of patients with painful diabetic neuropathy (i.e., patients with a Michigan Neuropathic Screening Instrument score of at least 3). The study will consist of a maximum 5-week screening/washout period (including 1-week of single-blind lead-in treatment) followed by a 17-week double-blind treatment phase (consisting of a 5-week titration period and 12 weeks of stable dosing). This will be followed by a 4-week withdrawal phase.

Patients will be randomized (1:1:1:1) to one of 4-double blind treatment groups (placebo, radiprodil, milnacipran hydrochloride, a combination of radiprodil and milnacipran hydrochloride).

The results from the above treatment regimen may surprisingly show that a combination of radiprodil and milnacipran hydrochloride can be used to safely and effectively treat painful diabetic neuropathy. The combination of radiprodil and milnacipran hydrochloride may provide synergistic results when compared to patients treated with radiprodil or milnacipran hydrochloride alone.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all values are approximate, and are provided for description.

The entire disclosures of all applications, patents and publications, cited above and below, are hereby incorporated by reference in their entirety.

What is claimed is:

1. A method of treating a disorder selected from diabetic neuropathic pain, post-herpetic neuralgia, chronic lower back pain, osteoarthritis and acute inflammatory pain comprising administering to a patient in need thereof a therapeutically effective amount of a compound of formula (I): 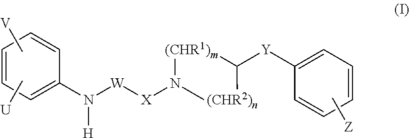
wherein
V and U are each independently
hydrogen, halogen, hydroxyl, cyano, nitro, amino, C₁-C₄ alkylamino optionally substituted by one or more halogen, arylamino optionally substituted by one or more halogen, aralkylamino optionally substituted by one or more halogen, C₁-C₄ alkylsulfonamido optionally substituted by one or more halogen, C₁-C₄ alkanoylamido optionally substituted by one or more halogen, arylsulfonamido, C₁-C₄ alkylsulfonyloxy, carboxyl, trifluoromethyl, trifluoromethoxy, C₁-C₄ alkyl-SO₂—NH—CH₂—, NH₂—(CH₂)₁₋₄—SO₂—NH—, NH₂—(CH₂)₁₋₄—(CO)—NH—, sulfamoyl, formyl, aminomethyl, hydroxymethyl, C₁-C₄ alkyl, C₁-C₄ alkoxymethyl, halogenated methyl, tetrazolyl,
or C₁-C₄ alkoxy, C₁-C₄ alkoxycarbonyl, C₁-C₆ alkanoyloxy, phenyl or C₁-C₄ alkoxy, each of which is optionally substituted by an amino group, or
neighboring V and U groups, together with one or more identical or different additional heteroatoms and/or —CH═ and/or —CH₂— groups optionally form a substituted 4-7 membered homo- or heterocyclic ring;
W and X are each independently —CO—, —CH₂— or —CH(C₁-C₄ alkyl)—, with the proviso that W and X can not simultaneously be methylene;
Y is —O—, C₁-C₄ alkylene, C₁-C₄ alkynylene, cycloalkylene, aminocarbonyl, —NH—, —N(C₁-C₄ alkyl)—, —CH₂O—, —CH(OH)— or —OCH₂—;
Z is hydrogen, halogen, nitro, amino, C₁-C₄ alkyl, C₁-C₄ alkoxy, cyano, trifluoromethyl, hydroxyl or carboxy;
R¹ and R² are each independently hydrogen or alkyl, or R¹ and R² together form an optionally substituted C₁-C₃ bridge and
n and m independently are 0-3, with the proviso that n and m can not simultaneously be 0;
and pharmaceutically acceptable salts or solvates thereof, or solvates of pharmaceutically acceptable salts thereof;
with the further provisos that
when Z is hydrogen, Y is —CH₂—, m and n are 2, R¹ and R² are hydrogen, W is —CO—, X is —CH₂— and V is hydrogen, then U is other than a 4-bromo substituent, and
when Z is hydrogen, Y is —CH₂—, m and n are 2, R¹ and R² are hydrogen, W and X are —CO— and V is hydrogen, then U is other than a 4-carboxyl or 4-ethoxycarbonyl substituent.

2. The method according to claim 1, wherein the compound of formula (I) is 2-[4-(4-fluoro-benzyl)-piperidine-1-yl]-2-oxo-N-(2-oxo-2,3-dihydro-benzoxazol-6-yl)acetamide, or a pharmaceutically acceptable salt thereof, solvate thereof, or a solvate of a pharmaceutically acceptable salt thereof.

3. The method according to claim 2, wherein the diabetic neuropathic pain is diabetic peripheral neuropathic pain.

4. The method according to claim 2, wherein the diabetic neuropathic pain is diabetic autonomic neuropathic pain.

5. The method according to claim 2, wherein the diabetic neuropathic pain is diabetic proximal neuropathic pain.

6. The method according to claim 2, wherein the diabetic neuropathic pain is diabetic focal neuropathic pain.

7. The method according to claim 2, wherein the disorder is post-herpetic neuralgia.

8. The method according to claim 2, wherein the disorder is chronic lower back pain.

9. The method according to claim 2, wherein the disorder is spinal cord injury.

10. The method according to claim 2, wherein the disorder is rheumatoid arthritis.

11. The method according to claim 2, wherein the disorder is osteoarthritis.

12. The method according to claim 2, wherein the disorder is acute inflammatory pain.

13. The method according to claim 2, wherein the therapeutically effective amount administered is from about 10 mg to about 150 mg.

14. The method according to claim 13, wherein the compound of formula (I) is administered in one, two, three or four divided daily doses.

15. The method according to claim 3, wherein the therapeutically effective amount administered is from about 10 mg to about 150 mg.

16. The method according to claim 4, wherein the therapeutically effective amount administered is from about 10 mg to about 150 mg.

17. The method according to claim 5, wherein the therapeutically effective amount administered is from about 10 mg to about 150 mg.

18. The method according to claim 6, wherein the therapeutically effective amount administered is from about 10 mg to about 150 mg.

19. The method according to claim 7, wherein the therapeutically effective amount administered is from about 10 mg to about 150 mg.

20. The method according to claim 8, wherein the therapeutically effective amount administered is from about 10 mg to about 150 mg.

21. The method according to claim 9, wherein the therapeutically effective amount administered is from about 10 mg to about 150 mg.

22. The method according to claim 10, wherein the therapeutically effective amount administered is from about 10 mg to about 150 mg.

23. The method according to claim 11, wherein the therapeutically effective amount administered is from about 10 mg to about 150 mg.

24. The method according to claim 12, wherein the therapeutically effective amount administered is from about 10 mg to about 150 mg.

25. The method according to claim 2, wherein the compound of formula (I) is adjunctively administered with an antidepressant, analgesic, muscle relaxant, anorectic, stimulant, antiepileptic drug, sedative/hypnotic and combinations thereof.

26. The method of claim 26, wherein the compound of formula (I) is adjunctively administered with milnacipran, gabapentin, pregabalin, pramipexole, 1-DOPA, amphetamine, tizanidine, clonidine, tramadol, morphine, tricyclic antidepressants, codeine, cambamazepine, sibutramine, amphetamine, valium, trazodone and combinations thereof.
